(19) United States
(12) Patent Application Publication
(10) Pub. No.: US 2008/0033356 A1
(43) Pub. Date: Feb. 7, 2008

(54) Device for the controlled piercing of an object and a method for operating the device

(76) Inventors: Jorn Kluge, Teltow (DE); Heinz Lehr, Berlin (DE)

Correspondence Address: SMITH PATENT OFFICE, 1901 PENNSYLVANIA AVENUE N W, SUITE 901, WASHINGTON, DC, 20006, US

(21) Appl. No.: 11/878,646

(22) Filed: Jul. 25, 2007

(30) Foreign Application Priority Data
Jul. 25, 2006 (EP) 06015462.2

(51) Int. Cl. A61M 5/32

(52) U.S. Cl. 604/117

(57) ABSTRACT

The invention relates to a device and a method for the controlled piercing of an object, particularly for the puncturing of a skin, the device including a drive unit configured for the purpose of supplying a drive force, a piercing system which comprises piercing means for piercing the object and which is coupled to the drive unit so that the drive force is initiated for a repetitive forward/return movement of the piercing means onto the piercing means, a measuring system that is configured for the purpose of determining, in a measuring operation mode, measuring values for a piercing depth of the piercing means into the object, and a control system which is coupled to the measuring system and the drive unit, and is configured for the purpose of setting, for an application operation mode, an extension amplitude for the repetitive forward/return movement of the piercing means, which is allocated to a pre-specified piercing depth which is derived from measuring values for the piercing depth as determined in the measuring operation mode.

The invention relates to a device for the controlled piercing of an object and a method for operating the device.

### BACKGROUND OF THE INVENTION

Devices for the controlled piercing of an object are used, for example, for the purpose of injecting an active substance into an object. Here, the term “active substance” is to be understood in very general sense. It can be preferably a medical or a cosmetic active substance. Included also are all types of vaccines as well as coloring materials such as, for example, tattoo coloring substances or coloring substances for permanent make-up. The substance can also be dermal fillers or substance used in a treatment known as carboxy therapy.

T- and B-cell immunity can be produced with the injection of special DNA-vaccines into the surface of the human skin. The antigens are suitable as vaccines against various tumors, influenza viruses as well as HIV (cf. Bins et al., Nature Medicine 1264, June 2005). During the vaccination process, the antigen is applied by means of a series of intracutaneous injections into the epidermis or the dermis. The objective in this case is to moisten a multiplicity of Langerhans cells which play an intermediary role in immunization processes. In a typical manner, some thousands of punctures with one or several injection needles are carried out on a surface of a few square centimeters, for example on the upper arm, in order to ensure that sufficient active substance has been applied for the vaccination process. A particularly significant factor here is that the active substance is applied to a certain area of the skin layers in order to obtain the desired effect. This depends in particular on a correct piercing depth of the piercing means.

With the use of the device for the controlled piercing of a skin as a tattoo or permanent make-up instrument, piercing is performed into the area of the derma. A piercing that goes even further can be envisaged for other applications.

Methods for measuring a piercing depth into an object, particularly a skin, are known as such in various embodiment forms. In the document WO 2004/080306, a measuring system for the measurement of a piercing depth is proposed for the purpose of performing sensory measurements where a contact ring on the skin serves as a first electrode which, together with a micro needle penetrating the skin, is to be used for impedance measurement. In this case, the impedance change as a function of the penetration depth into the skin serves as a measuring value. However, depending on the type of skin and the type of electrode, both the absolute value of the impedance as well as the reaction of the impedance as a function of the penetration depth is very varied indeed. Subsequently, such a measuring method for the vaccination process must inevitably fail. It was proposed to apply an additional reference electrode on the skin for standardization purposes in order to at least rule out the unsteadiness of the skin contact.

### SUMMARY OF THE INVENTION

It is an object of the invention to provide a device for the controlled piercing of an object, particularly for the controlled puncturing of a skin, and to provide a method for the operation of the device in which the usage characteristics are improved for the user.

This object is solved according to the invention by means of a device according to the independent claim 1 as well as a method according to the independent claim 21.

The invention makes it possible for the user to carry out the piercing of an object in a defined manner with simultaneous adherence to application pre-specified data when using the device. The device enables the user to determine a piercing depth of the piercing means in the measuring operating mode. Depending on the measured piercing depth, the device can be controlled in such a way that an extension amplitude of the piercing means is set which, on its part, ensures a pre-specified piercing depth. For the measurement of the piercing depth in the measuring operating mode, random measuring methods for determining the piercing depth can be used which are known, for example, for the measurement of the piercing depth of a skin. Therefore, the device combines the advantages of an exact measurement of the piercing depth of the piercing means with the specific controlling of the extension amplitude of the forward/return movement of the piercing means in the application operating mode. Preferably, the forward/return movement of the piercing means is carried out with a frequency between about 30 Hz and about 250 Hz, more preferably between about 50 Hz and about 200 Hz.

It is envisaged with an advantageous embodiment of the invention that a reservoir is formed with a liquid containing an active substance where said liquid is connected to the piercing means by means of a fluid connection, by way of which the liquid in at least one application operating mode for controlled active substance discharge makes its way to the piercing means. In this way, the possibility is created for carrying out the controlled discharge of a liquid with an active substance in a defined manner depending on a piercing depth of the piercing means.

A purposeful development of the invention envisages a configuration of the measuring system in order to determine, in the measuring operating mode, measuring values for a piercing depth of the piercing means into the object in accordance with at least one of the following measuring modes: single piercing measurement and measurement of several piercings with repetitive forward/return movements of the piercing means.

A preferred further development of the invention envisages that the fluid connection is a pulsed-operable fluid connection which is configured for the purpose of supplying the liquid in partial volumes from the reservoir to the piercing means depending on a movement frequency of the repetitive forward/return movements of the piercing means.

For a purposeful embodiment of the invention it can be envisaged that the piercing means are routed through a breakthrough in a wall of the reservoir, in which the piercing means are arranged for the repetitive forward/return movement.

An advantageous embodiment form of the invention envisages a pumping system configured for the purpose of pumping the liquid from the reservoir to the piercing means.

A preferred development of the invention envisages that the control system is coupled to the pump system and is configured for the purpose of discharging a pump impulse to the pump system, having an excitation effect on the pump system, which impulse is allocated to a pre-specified extension amplitude of a extended position of the piercing means with the repetitive forward/return movements of the piercing means.

For an advantageous embodiment form of the invention it can be envisaged that the control system is configured for the purpose of discharging the pump impulse to the pump system if the pre-specified amplitude corresponds to a maximum extended position of the piercing means.

A further development of the invention can envisage that the pump system comprises a micro pump.

A preferred further development of the invention envisages that the fluid connection comprises a channel in the piercing means which leads to a discharge opening formed at the piercing means.

It can be envisaged for a purposeful embodiment of the invention that the measuring system is an impedance measuring system for the measurement of an impedance of the object.

An advantageous embodiment form of the invention envisages that the piercing means comprise one or several measurement electrodes, which are coupled to the measuring system.

A development of the invention preferably envisages that several measurement electrodes are each formed by a needle enclosed by the piercing means.

For an advantageous embodiment of the invention, it can be envisaged that a measurement electrode is formed by means of the piercing means and a measurement counter electrode by a casing part.

A further development of the invention can envisage that the piercing system is formed in a disposable module, which is detachable from the drive unit. A piercing system comprising such disposable module is described, for example, in U.S. Pat. No. 6,345,553 which is incorporated here by reference.

A preferred further development of the invention envisages that, by means of an electric impulse generator, which is electrically connected to the piercing means and is configured, for the purpose of transmitting an electric impulse to the piercing means at a pre-specified point of time. An amplitude of the electric impulse is controllable. With the use of the device for skin puncturing, this can be selected to such a large size that a skin stimulation up to a coagulation of the skin is achieved.

For a purposeful embodiment of the invention it can be envisaged that the drive unit comprises a conversion mechanism which is configured for the purpose of converting a turning movement of the electric motor into a repetitive thrust movement to be applied to the piercing means.

An advantageous embodiment form of the invention envisages that the conversion mechanism comprises a wobble disk or plate arrangement in which a wobble disk is coupled to a drive shaft of the electric motor and a coupling mechanism is coupled to the wobble disk, where the coupling mechanism is configured for the purpose of initiating the repetitive thrust movement onto the piercing means. Conversion mechanism comprising a wobble disk or plate arrangement is described, for example, in US 2005/0010236 A1 which is incorporated here by reference.

A preferred development of the invention envisages that the coupling mechanism has a tappet that is coupled to the wobble disk, which tappet is in a linear arrangement.

For an advantageous embodiment of the invention it can be envisaged that the reservoir can be formed in a piercing means nozzle. In a preferred embodiment, the piercing means nozzle is incorporated in the casing part. In combination therewith or alternatively thereto, an embodiment of the invention envisages that the reservoir is formed by means of a reservoir tank that is detachably attached to the hand-held device and is replaceable as a result, whether it be used for replenishing or for changing the reservoir volume, which reservoir tank is connected to a partial section of the fluid connection, the reservoir tank being, for example, plugged on a connection piece or screwed onto same. The reservoir tank is preferably provided as a disposable tank. Alternatively, the reservoir may be provided also in other parts of the device. Even though the envisaging of a reservoir is preferred, such a reservoir can be dispensed with in a simplified embodiment. The liquid to be discharged accesses the piercing means by immersing them in a liquid volume. This immersion is repeated several times during the usage of the device. When immersing, the liquid to be discharged is distributed at least along the outer surface of the piercing means.

Furthermore, and where the invention is concerned, preferred embodiments of the method can be established. These are explained as follows in greater detail.

For an embodiment form of the invention, it is envisaged that the liquid is conducted from a reservoir to the piercing means by way of a liquid connection.

A development of the invention envisages that the piercing depth in the measuring operation mode is measured according to at least one of the following measuring methods: single-piercing measurement and measurement of several piercings with repetitive forward/return movements of the piercing means.

A further development of the invention can envisage that the measuring operation is aborted after the pre-specified piercing depth has been determined from the measured measuring values.

A preferred further development of the invention envisages that the liquid is supplied by way of a pulsed fluid connection in partial volumes from the reservoir to the piercing means in dependence of a movement frequency of the repetitive forward/return movements of the piercing means.

It can be envisaged with a purposeful embodiment of the invention that the liquid, under the use of adhesion forces, is conducted along a surface section of the piercing means, where the piercing means are conducted through a breakthrough in a wall of the reservoir, in which the piercing means are routed for the repetitive forward/return movements.

An advantageous embodiment of the invention envisages that the liquid is pumped by means of a pump from the reservoir to the piercing means.

A preferred development of the invention envisages that the control system is coupled to the pump system and transmits a pump impulse to the pump system, having an excitation effect on the pump system, which impulse is allocated to a pre-specified extension amplitude of a extended position of the piercing means with the repetitive forward/return movements of the piercing means.

It can be envisaged for an advantageous embodiment of the invention that the control system transmits the pump impulse to the pump system if the pre-specified extension amplitude corresponds to a maximum extended position of the piercing means.

A further development of the invention can envisage that, in the measuring operation mode, the measuring values for the piercing depth are detected as impedance measuring values of the object.

A preferred further development of the invention envisages that, by means of an electric impulse generator which is electrically connected to the piercing means, an electric impulse is transmitted to the piercing means at a pre-specified point of time, through which a high electric field strength is produced at the piercing means, where an impulse discharge point of time of a further pre-specified extension amplitude in a further extended position of the piercing means is allocated for the repetitive forward/return movements of the piercing means, where the further pre-specified extension amplitude selectively coincides with the pre-specified extension amplitude. It can be envisaged that the pre-specified extension amplitude and/or the further pre-specified extension amplitude correspond to the extension amplitude allocated to the pre-specified piercing depth. Even if the electric impulse generator is preferably integrated in the device for the controlled liquid discharge, a device can thus be envisaged as an alternative in which the impulse generator is combined with the components for piercing depth measurement and for the setting of an extension amplitude dependent hereof, without the formation of a liquid discharge and, subsequently, the instruments used for this. In an analogous manner, and in an impulse discharge operating mode, electric impulses are transmitted to the repeatedly moving piercing means.

With a purposeful embodiment of the invention it can be envisaged that, in the drive unit with a conversion mechanism, a turning movement of an electric motor is converted into a repetitive thrust movement to be applied to the piercing means.

The method or the device may be used for the purpose of injecting an active substance into an object. Here, the term “active substance” is to be understood in very general sense. It can be preferably a medical or a cosmetic active substance. Included also are all types of vaccines as well as coloring materials such as, for example, tattoo coloring substances or coloring substances for permanent make-up. The substance can also be dermal fillers or substance fused treatment known as carboxy therapy.

### DESCRIPTION OF PREFERRED EMBODIMENT FORMS OF THE INVENTION

The invention is described as follows in greater detail on the basis of preferred embodiment forms with reference to the Figures of a drawing. The Figures show the following:

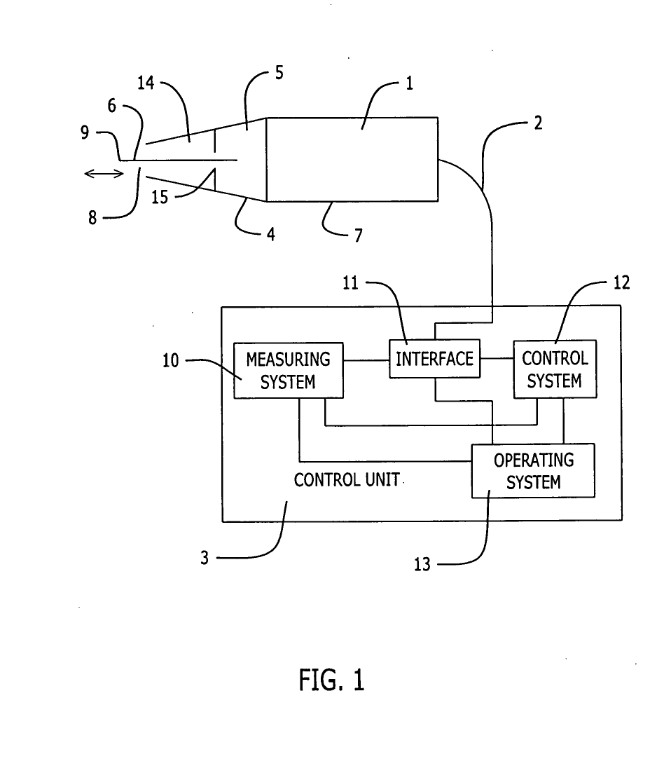

FIG. 1 a schematic illustration of a device for the controlled discharge of a liquid containing an active substance;

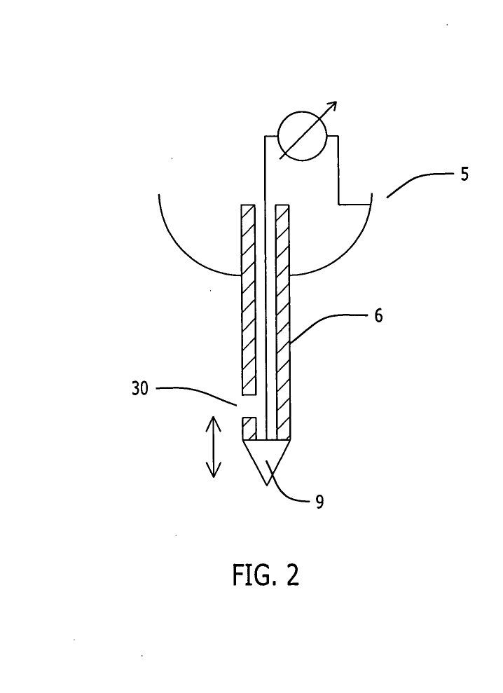

FIG. 2 a schematic illustration of a piercing tool with a measuring system coupled thereto for measuring the piercing depth of a piercing means;

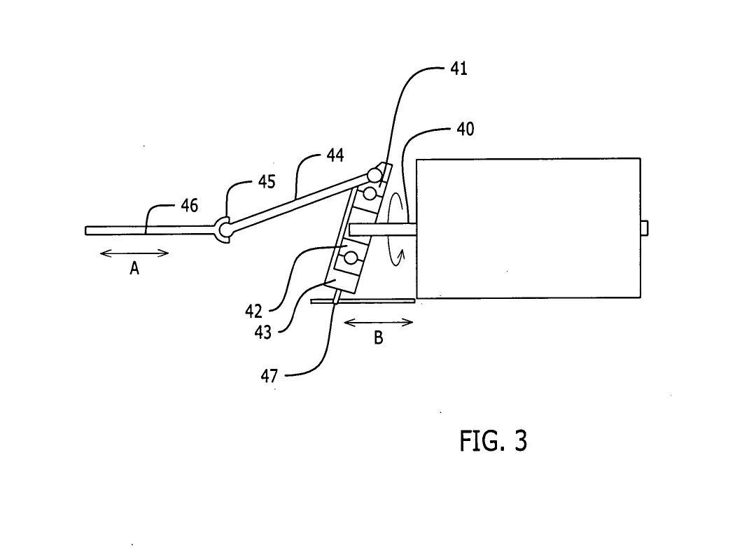

FIG. 3 a wobble disk drive in a cross-sectional view;

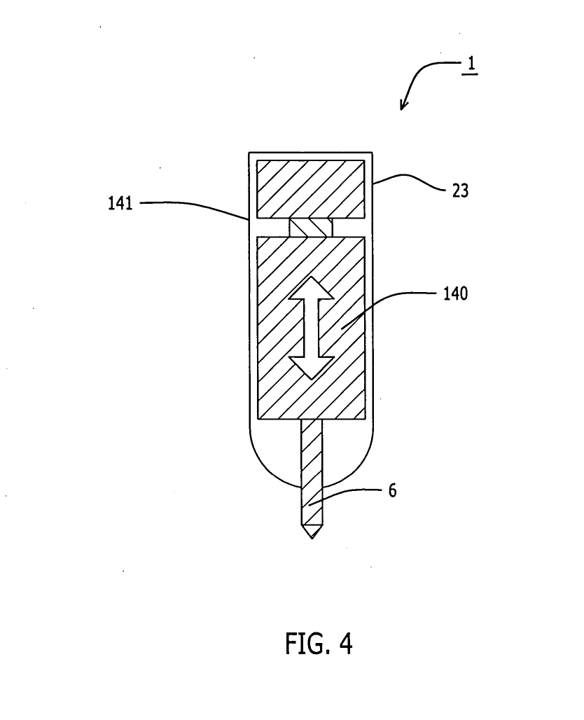

FIG. 4 a schematic illustration of a hand-held device with a piercing tool and a drive unit;

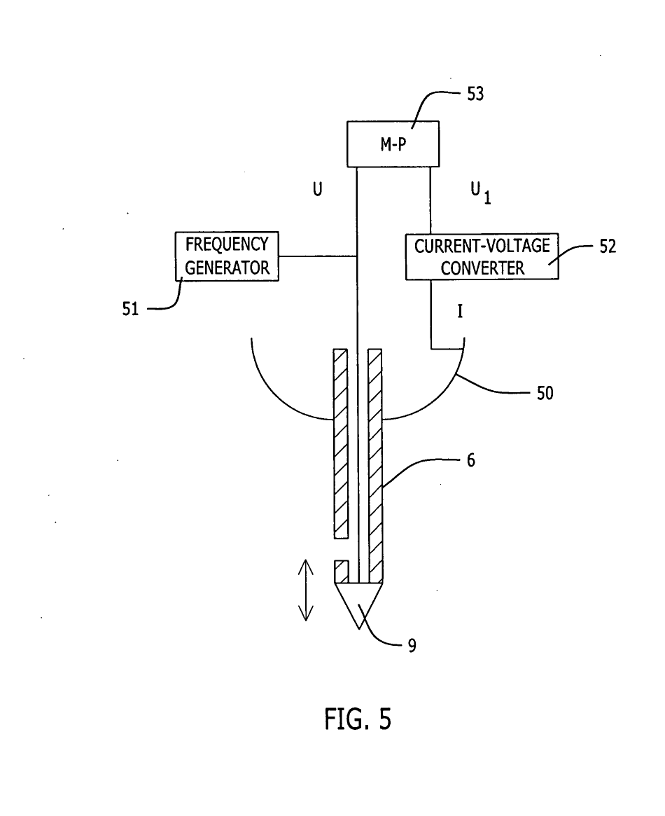

FIG. 5 a schematic illustration of a piercing tool with a measuring system coupled hereto for measuring a piercing depth of a piercing means; and

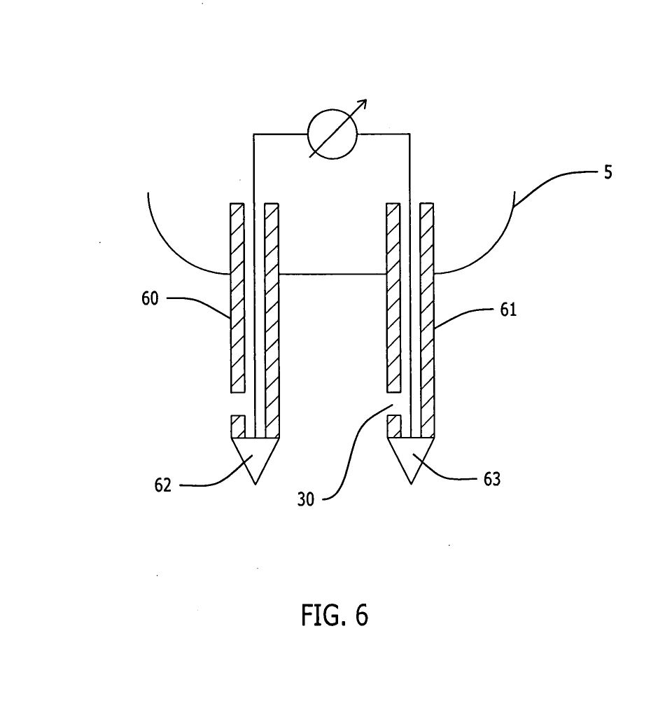

FIG. 6 a schematic illustration of a further piercing tool with a measuring system coupled hereto for measuring the piercing depth of a piercing means.

FIG. 1 shows a schematic illustration of a device for the controlled piercing of an object, preferably with the inclusion of a controlled discharge of a liquid containing an active substance. A hand-held device 1, which is held in the hand of a user during the discharge of the liquid, is connected to a control unit 3 by way of a connecting cable 2 which can also be executed as a cordless connection in an alternative embodiment. On the one hand the hand-held device 1 is supplied with electric operating voltage by way of the connecting cable 2, where said voltage can normally be regulated, and on the other hand electronic data are exchanged between the hand-held device 1 and the control unit 3. The latter comprise in particular measuring data and control data.

The hand-held device 1 comprises as a basic component a piercing tool 4 with a holder 5 in which a piercing means 6 is located in a slidable manner. The piercing means 6 is either a single needle or a needle system with two or several needles. The piercing tool 4 is coupled to a drive unit 7 which serves the purpose of moving the piercing means 6 forwards and backwards repeatedly. In this case the piercing means 6 moves through an opening 8, wherein a tip 9 of the piercing means 6 can be located either in front of or behind the opening 8 in a completely retracted position.

An extension amplitude of the piercing means 6, meaning, the length of a section of the piercing means 6 outside of the opening 8 in a completely extended position of the piercing means 6, can be set automatically in such a way that either a stroke of the piercing means 6 or a position of the opening 8 in the longitudinal direction of the piercing means 6 is changed. The latter is achieved with a displacement of the holder 5. A combination of these two setting options can also be envisaged for the definition of the extension amplitude.

In the control unit 3, a measuring system 10 is formed which is coupled by way of an interface 11 to the connecting cable 2 and, in this way, is coupled to the hand-held device 1. In the schematic illustration in FIG. 1, the measuring system 10 is integrated in the control unit 3. It is taken for granted that elements of a measuring arrangement comprising the measuring system 10 in FIG. 1 can also be integrated in the hand-held device 1, for example electrodes for a contact with the object to be measured. With the measuring arrangement, the piercing depth of the piercing means 6 into the object is detected. This is explained below in detail.

The control unit 3 comprises furthermore a control system 12, which is connected to the interface 11 and the measuring system 10. The control system 12 particularly serves the purpose of setting a pre-specified extension amplitude of the piercing means 6 in an application designated, for example, as an active substance discharge operating mode, whereby a pre-specified piercing depth is realized. The extension amplitude set in this way can be larger or smaller than the piercing depth measured in the measuring operating mode, or can correspond to this.

The size of the extension amplitude set in the active substance operating mode is fixed by the control system 12 in dependence of the measuring values for the piercing depth in a preceding measuring operating mode. In this case, a user of the control unit 3 can define any required pre-specified details for the liquid discharge by way of an operating system 13, such as a keyboard or a contact-sensitive display, for example the selection of a certain skin layer into which the liquid with the active substance is to be injected. The control system 12 is configured on the basis of such a pre-specification for the purpose of setting an extension amplitude, with due consideration of the measuring results in the measuring operating mode, where said extension amplitude corresponds to a piercing depth to be allocated to the user setting. For example, this can be done in such a way that a certain measuring result for the piercing depth is allocated to a skin layer selected by the user. If this measuring result then appears in the measuring operating mode during the determination of the piercing depth at that moment, the control system 12 recognizes the extension amplitude pertaining hereto and sets this for the active substance discharge operating mode.

The movement of the piercing means is initiated by a periodic stroke movement that is generated by means of the drive system 7, which comprises an electric motor for producing a drive movement.

In one embodiment, the holder 5 is made from metal and has a dome-shaped contour. In operation, the hand-held device 1 is placed onto the object to be pierced, for example the skin surface, and is moved vertically in contact with the surface of the object in such a way that, with the oscillatory stroke movement of the piercing means 6 coming from the hand-held device 1, the liquid is applied by means of numerous piercings in randomly large surfaces of the object. In the simplest case, the liquid is discharged by a movement of the piercing means 6, where the piercing means 6 moves forwards and backwards in a reservoir 14 which is filled with the liquid to be discharged. The reservoir 14 can be filled with capillary forces by immersing the holder 5 at least partially in a liquid volume. A rear-side opening 15 is sealed off by means of a membrane seal enveloping the piercing means 6.

In this case, the output of the liquid is effected because of the adhesion-controlled entrainment of the liquid through the piercing means 6. However, this type of output of the liquid is less reproducible as the liquid is partially wiped off when the piercing means 6 penetrates the object surface. It is therefore unclear as to what volume of the active substance has actually been placed at the “correct” location. In a further type the liquid discharge is effected at the time of reaching a maximum extension amplitude when the piercing means 6 has reached a required piercing depth, by transmitting an ejection impulse to the reservoir 14 with the discharging liquid, for example by means of a micropump (not shown), which is coupled to the reservoir 14. In an alternative embodiment, the reservoir can be formed outside of the holder 5, for example as an external tank located at the hand-held device 1.

The dome-shaped metal surface of the holder 5 forms a measurement electrode, whereas the tip 9 acts as a counter electrode. The piercing depth is measured in the measurement operating mode by means of an impedance measurement. Depending on the characteristics of the object in the piercing area, certain impedance leaps can occur which show characteristic piercing depths which again, on their part, are to be allocated to certain layers of the object. The occurrence of such a characteristic measuring value can then be used for the purpose of fixing a pre-specified extension amplitude for the active substance discharge operating mode, wherein the extension amplitude allocated to the characteristic measuring value can be increased or decreased for this purpose.

The piercing movement of the piercing means is carried out with a piercing frequency between about 30 Hz and about 250 Hz, more preferably between about 50 Hz and about 200 Hz. With simultaneous translatory movement of the hand-held device 1 over the surface of the object, the liquid is applied very quickly with a large number of injections so that, for example, a vaccination lasts only a few seconds. An essential factor for a secure input of the liquid in this case is the precise setting of the piercing depth which is effected by the measurement in the measuring operation mode as well as a controlled setting of the stroke amplitude by means of the control system 12.

Very small piercing depths are frequently desired, for example approx. 50 μm to a few 100 μm. For this reason, the point of time of the liquid discharge as well as the secure handling of the hand-held device 1 are critical factors for a reliable discharging process. If additional mass bodies are moved for the energetic implementation of the stroke movement of the piercing means 6, except for the piercing means 6 itself, this leads to vibrations in a longitudinal and possible transverse direction to the stroke movement. As a result, no secure and reliable active substance input is possible in the required depth. Examples for such less suitable drive elements, depending on the circumstances, in the drive unit 7 include screw-type drives, crank drives, pneumatic and mechanical drives for moving the piercing means 6. Because of the moving large masses in addition to the piercing means 6 itself, these drives can be executed with frequently less sufficient low-vibration levels. The piercing frequencies are limited for this reason. Moreover, these drives have large constructional space requirements because of their mode of movement. Therefore, they are a possible obstacle for a secure and reliable handling of the hand-held device 1.

In a preferred embodiment, a wobble disk drive is used with which a rotational movement is converted into a repetitive forward/return movement for driving the piercing means 6. FIG. 3 shows a wobble disk drive in a cross-sectional view.

FIG. 3 shows a schematic illustration of a drive mechanism where, on a drive shaft 40, a ball-bearing 41 with an inner ring 42 and a freewheeling outer ring 43 is mounted in a slanted position on the drive shaft 40. At the outer ring 43, a connecting rod 44 is supported in position which is connected to a thrust rod 46 by way of a joint 45. When the drive shaft 40 is turned, the ball bearing 41 performs a wobble movement which is converted into a linear drive movement for the piercing means 6 in the direction of the arrow A with the help of the connecting rod 44 and the thrust rod 46. In this case, the outer ring 43 is held in position with the help of an anti-turn protection element 47 which moves forwards and backwards in the direction of arrow B.

With the movement of the freewheeling outer ring 43, a fixed extension amplitude of the piercing means 6 is produced, 2 mm for example. The fixed extension amplitude can be effected variably for the piercing operation by means of a displacement of a drive unit 140 with the wobble disk drive in the casing 141 of the hand-held device 1 relative to the opening 8, as shown schematically in FIG. 4. Therefore, the displacement of the drive unit 140 in the casing 141 establishes the setting of the desired piercing depth. The relative movement of the drive unit 140 can take place, for example, by means of the rotator drive 23 at the upper end of the hand-held device 1 (FIG. 4). The setpoint pre-specification for the piercing depth results from the piercing depth measurement.

At the beginning of the overall process for the controlled discharge of the liquid in the measuring mode, the extension amplitude of the piercing means 6 is selected very small at first and is then increased in small increments up to the desired piercing depth, indicated upon reaching certain measuring values, for example an impedance leap. Some piercing actions are required during the gradual increase of the amplitude up to the setpoint piercing depth. An impedance measurement takes place at every piercing action. The measuring values are stored in a signal processor allocated to the measuring system 10. If the piercing means has penetrated to a desired depth, a characteristic measuring value appears, for example an expected impedance value. Then, the extension amplitude obtained at that point can be adopted for the further process and, in the following procedure, can be used as a fixed point for the piercing depth.

FIG. 5 shows a schematic illustration of a piercing tool with a measuring system coupled to it for the measurement of a piercing depth of piercing means.

Various electrode variants for determining the piercing depth are envisaged depending on the equipment design. In a multiple needle system, for example, it can be envisaged that only a part of the needles used for piercing purposes is also included as an electrode in the measurement. The impedance between at least two electrodes is determined which are formed in the embodiment in FIG. 5 by means of the tip 9 of the piercing means 6, this being a hollow needle, and a section 50 of the holder 5. Furthermore, a frequency generator 51 is envisaged. For measurement purposes, an alternating voltage amplitude of maximum 200 mV is applied between the electrodes. The current flowing between the electrodes enters a signal processor 53 by way of a current-voltage converter 52 as a current-proportional voltage signal, and this processor 53 performs the evaluation and storage of the impedance measuring value together with the voltage signal. The frequency of the alternating voltage lies in the range of approx. 50 kHz to approx. 100 kHz. Within this frequency range, a safe and reliable discrimination is particularly possible between skin tissue and blood when active substance is injected into the skin.

In the embodiment shown in FIG. 2, the piercing means 6 executed as a single needle (hollow needle) is used as an electrode, particularly in the area of the tip 9. The metallic holder 5 is used as a counter electrode and for the electric contact with the object for impedance measurement. During the piercing action, current and voltage are measured as a function of the piercing depth. If the piercing means 6 penetrates the object, a characteristic impedance results for the object in the piercing zone because of the current and voltage measurement. An outlet opening 30 for the liquid is formed with a defined spacing above the tip 9. The outlet opening 30 is in fluid connection with the reservoir (not shown) for the liquid to be extracted. In the measurement operating mode, and for the determination of the setpoint piercing depth, not absolute measuring values are examined but rather a relative change of the measuring values as a result of the appearance of characteristic measurement value leaps. As known, for example, the skin contact as well as the precision of the impedance measurement depends very much on the surface condition of the skin, for example, as a result of the excretion of perspiration. Examinations have shown that, for example, an absolute measurement of the piercing depth into the skin is frequently difficult as a result of the constantly varying skin parameters.

According to FIG. 6, two rigidly joined needles 60, 61 are used as piercing means in another embodiment example, where both penetrate the skin together, wherein electrodes 62, 63 for the impedance measurement are each formed at the tip. More than two piercing needles can also be used. Compared with the previous embodiment example, the advantage of the arrangement shown in FIG. 6 lies in the fact that several vaccination injections can be given at the same time so that the vaccination process can be completed quicker. Furthermore, the impedance measurement can be carried out between several needle electrodes which penetrate the object parallel and simultaneously and are not dependent on the object contact of an electrode. As a result, better and reliable measurement values are achievable for the piercing depth.

In the same manner as with the measurement with one needle, the penetration depth of the needles are increased at the beginning of the vaccination process up to the characteristic leap of the impedance. The extension amplitude of the piercing movement is then “frozen” for the further vaccination process where again the active substance is administered by means of multiple injections. In this case, several needles are available for the discharge of the active substance. For this reason, the vaccination time is reduced by the factor of the number of needles compared with the single-needle solution.

The discharge of active substance for the various embodiment forms can be carried out in an uncomplicated manner by the immersion of the piercing means into a reservoir for the liquid during the return movement. The entrainment of the liquid for discharge takes place in the simplest case by means of adhesion of the liquid at the needle, particularly in grooves, recesses or transverse bores of the piercing means. However, the use of needles with a smooth surface can also be envisaged. In another embodiment, the piercing means are executed as hollow needles, an outlet opening for the liquid preferably being provided at the side in order to prevent a blockage of the hollow body (cf. FIG. 2, above). For this type, the needle movement and the point of time of liquid discharge are solidly coupled.

In a purposeful embodiment the liquid discharge is effected upon reaching a maximum penetration depth of the piercing means, for example by means of a pressure surge of a “micro-pump” (not shown). For example, a periodic pressure excitation of a liquid enclosed within an elastically deformable reservoir tank can be envisaged, for example with a contracting piezo ring which exerts pressure onto the reservoir synchronously with the forward movement of the piercing means, or a tappet that produces the pressure excitation and which is coupled to the piercing means movement.

With the help of the device described here, active substances can be injected specifically into an object, into the skin for example. Langerhans cells are located in the prickle cell layer (stratum spinosum), at the lower end of the epidermis. In order to bring these into contact with the vaccine in an optimal manner, the active substance must be released in the immediate vicinity of the Langerhans cells and under no circumstances in the derma located below the epidermis. If an injection needle penetrates too deeply during a piercing action, there is the danger that the active substance enters the papillary derma and subsequently into the blood, or is routed into the collagen bundle of the derma and, as a result, cannot develop its effectiveness. Exactly this can be avoided with the device as described above.

The features of the invention as disclosed in this description, in the claims and in the drawing can be of significance both individually as well as in random combination for the realization of the invention in its various embodiment forms.

This application is based on European Patent Application No. 06015462.2 filed on Jul. 25, 2006, and the contents of which are incorporated hereinto by reference.

1. Device for the controlled piercing of an object, particularly the puncturing of a skin, with a drive unit that is configured for the purpose of supplying a drive force, and a piercing system comprising piercing means for piercing the object and which is coupled to the drive unit, so that the drive force for a repetitive forward/return movement of the piercing means is initiated and applied to the piercing means, characterized by
a measuring system which is configured for the purpose of determining measuring values for a piercing depth of the piercing means into the object in a measuring operation mode, and
a control system coupled to the measuring system and to the drive unit and which is configured for the purpose of setting, for an application operation mode, an extension amplitude for the repetitive forward/return movement of the piercing means which are allocated to a pre-specified piercing depth that is derived from the measuring values determined in the measuring operation mode for the piercing depth.

2. The device according to claim 1, wherein a reservoir is formed with a liquid containing an active substance where said reservoir is connected to the piercing means by means of a fluid connection, by way of which the liquid makes its way to the piercing means at least in the application operation mode for the controlled discharge of active substance.

3. The device according to claim 1, wherein the measuring system is configured for the purpose of determining, in the measuring operation mode, measuring values for a piercing depth of the piercing means in an object according to at least one of the following measuring modes: single-piercing measurement and measurement of several piercings for repetitive forwards/return movements of the piercing means.

4. The device according to claim 2, wherein the fluid connection is a pulsed operable fluid connection which is configured for the purpose of supplying the liquid in partial volumes from the reservoir to the piercing means depending on a movement frequency of the repetitive forward/return movements of the piercing means.

5. The device according to claim 2, wherein the piercing means are routed through a breakthrough in a wall of the reservoir in which the piercing means for the repetitive forward/return movements of the piercing means are routed.

6. The device according to claim 2, further comprising:
a pump system that is configured for the purpose of pumping the liquid from the reservoir to the piercing means.

7. The device according to claim 6, wherein the control system is coupled to the pump system and is configured for the purpose of transmitting a pump impulse, having an excitation effect on the pump system, to the pump system which is allocated to a pre-specified extension amplitude of a extended position of the piercing means during the repetitive forward/return movements of the piercing means.

8. The device according to claim 7, wherein the control system is configured for the purpose of transmitting the pump impulse to the pump system if the pre-specified amplitude corresponds to a maximum extended position of the piercing means.

9. The device according to claim 6, wherein the pump system comprises a micro-pump.

10. The device according to claim 2, wherein the fluid connection comprises a channel in the piercing means, which leads to a discharge opening formed at the piercing means.

11. The device according to claim 1, wherein the measuring system is an impedance measuring system for measuring an impedance of the object.

12. The device according to claim 1, wherein the piercing means comprise one or several measuring electrodes coupled to the measuring system.

13. The device according to claim 12, wherein several measuring electrodes are formed in each case by a needle enclosed by the piercing means.

14. The device according to claim 12, wherein a measuring electrode is formed by means of the piercing means and a measuring counter electrode is formed by a casing part.

15. The device according to claim 1, wherein the piercing system is formed in a disposable module detachable from the drive unit.

16. The device according to claim 1, further comprising:
an electric impulse generator which is connected electrically to the piercing means and which is configured for the purpose of transmitting an electric impulse to the piercing means at a pre-specified point of time.

17. The device according to claim 1, wherein the drive unit comprises a conversion mechanism which is for the purpose of converting a turning movement of the electric motor into a repetitive thrust movement to be applied to the piercing means.

18. The device according to claim 17, wherein the conversion mechanism comprises a wobble disk arrangement in which a wobble disk is coupled to a drive shaft of the electric motor and a coupling mechanism is coupled to the wobble disk, where the coupling mechanism is configured for the purpose of initiating the repetitive thrust movement onto the piercing means.

19. The device according to claim 18, wherein the coupling mechanism has a tappet that is coupled to the wobble disk, which tappet is positioned in a linear arrangement.

20. The device according to claim 2, wherein the reservoir is formed in a piercing means nozzle.

21. Method for operating a device for the controlled piercing of an object in which piercing means enclosed by a piercing system are put into a repetitive forward/return movement for piercing an object by means of a drive unit which supplies a force, characterized in that, in a measuring operation mode by means of a measuring unit, measuring values for a piercing depth of piercing means in an object are measured and, by means of a control system that is coupled to the measuring unit and the drive system, an extension amplitude for an application operation mode is set for the repetitive forwards/return movement of the piercing means, which amplitude is allocated to a pre-specified piercing depth which is derived from measuring values for the piercing depth determined in the measuring operation mode.

22. The method according to claim 21, wherein in at least one application operation mode for the controlled discharge of active substance, which follows the measuring operation mode, a liquid containing an active substance is discharged to the piercing means.

23. The method according to claim 21, wherein the liquid from a reservoir is discharged to the piercing means by way of a fluid connection.

24. The method according to claim 21, wherein the piercing depth in the measuring operation mode is measured according to at least one of the following measuring modes: single-piercing measurement and measurement of several piercings for repetitive forward/return movements of the piercing means

25. The method according to claim 21, wherein the measuring operation mode is aborted after the pre-specified piercing depth has been determined from the measured measuring values.

26. The method according to claim 23, wherein the liquid is supplied by way of a pulsed fluid connection in partial volumes from the reservoir to the piercing means in dependence of a movement frequency of the repetitive forward/return movements of the piercing means.

27. The method according to claim 23, wherein the liquid, with the usage of adhesion forces, is routed along a surface section of the piercing means where the piercing means are routed through a wall of the reservoir in which the piercing means for the forward/return movements of the piercing means are guided.

28. The method according to claim 23, wherein the liquid is pumped to the piercing means by means of a pump system.

29. The method according to claim 27, wherein the control system is coupled to the pump system and transmits a pump impulse to the pump system, having an excitation effect on the pump system, which impulse is allocated to a pre-specified extension amplitude of an extended position of the piercing means during the repetitive forward/return movement of the piercing means.

30. The method according to claim 29, wherein the control system transmits the pump impulse to the pump system if the pre-specified extension amplitude corresponds to a maximum extended position of the piercing means.

31. The method according to claim 21, wherein in the measuring operation mode, in the measuring values are detected for the piercing depth as impedance measuring values of the object.

32. The method according to claim 21, wherein, by means of an electric impulse generator which is electrically connected to the piercing means, an electric impulse is transmitted to the piercing means at a pre-specified point of time, through which a high electric field strength is produced at the piercing means, where an impulse discharge time of a further pre-specified extension amplitude in a further extended position of the piercing means is allocated for the repetitive forward/return movements of the piercing means, where the further pre-specified extension amplitude selectively coincides with the pre-specified extension amplitude.

33. The method according to claim 21, wherein, in the drive unit, a turning movement of the electric motor is converted into a repetitive thrust movement to be initiated and applied to the piercing means with a converting mechanism.
